(19) United States
(12) Patent Application Publication
(10) Pub. No.: US 2025/0352079 A1
(43) Pub. Date: Nov. 20, 2025

(54) METHODS AND APPARATUS FOR DETECTING ABNORMAL TISSUE AND OTHER FOREIGN MATTER IN A BODY

(71) Applicant: BOARD OF TRUSTEES OF NORTHERN ILLINOIS UNIVERSITY, DEKALB, IL (US)

(72) Inventor: S. Mohammad J. MOGHIMI, Glassboro, NJ (US)

(21) Appl. No.: 19/284,897

(22) Filed: Jul. 30, 2025

Related U.S. Application Data
(63) Continuation of application No. PCT/US24/13319, filed on Jan. 29, 2024.
(60) Provisional application No. 63/442,018, filed on Jan. 30, 2023.

(51) Int. Cl. A61B 5/0531; A61B 5/00

(52) U.S. Cl. CPC A61B 5/0531; A61B 5/6821; A61B 5/6833; A61B 2562/164

(57) ABSTRACT

This disclosure pertains to methods, apparatus, systems, and techniques for non-invasively detecting abnormal biological tissue and other abnormal matter in a body.

### CROSS REFERENCE TO RELATED APPLICATIONS

This application is a continuation of International Application No. PCT/US24/13319, file Jan. 29, 2024, which claims the benefit of U.S. Provisional Patent Application No. 63/442,018 filed Jan. 30, 2023, each of which are incorporated herein by reference in its entirety.

### FIELD OF THE INVENTION

This disclosure pertains to methods, apparatus, systems, and techniques for non-invasively detecting abnormal biological tissue and other foreign matter in a body.

### BACKGROUND

The American Cancer Society estimated that 97,610 individuals will be diagnosed with melanoma in 2023, and the mortality rate will be 7,990 in the same year. If melanoma is detected in the early stage of development (stage 0, I, and II) and the tumor is localized, the 5-year survival rate is as high as 98% according to the Melanoma Research Alliance.

Thus, early detection of melanoma is essential to increasing the chances of successful treatment and to reduce the mortality rate.

Existing procedures for diagnosis rely on biopsy and imaging of the cells in the tumor, which is invasive and requires intervention by highly skilled specialists.

Another procedure is the non-invasive method of imaging cells with reflectance confocal microscopy (RCM). This procedure also requires intervention by specialists in hospitals and clinics, which is expensive and often delays the detection of melanoma.

Clinical studies have shown that electrical impedance spectroscopy (EIS) is able to distinguish melanoma tumors from healthy skin tissues in the vicinity of the tumors, perhaps due to abnormal cell density, blood flow, and cell shape. See J. Malvehy et al., “Clinical performance of the Nevisense system in cutaneous melanoma detection: An international, multicentre, prospective and blinded clinical trial on efficacy and safety,” Br. J. Dermatol., vol. 171, no. 5, pp. 1099-1107, 2014, and F. M. Thesis, “Lithuanian University of Health Sciences Electrical Impedance Spectroscopy (EIS)—An Overview of a New Method in Melanoma Diagnosis—,” 2020. The Nevisense system, however, relies on a special gold electrode having “high precision micro-structures” (www.scibase.com/our-electrodes/) described as penetrating “pins” or “needles” (Sarac, E., et al., Diagnostic Accuracy of Electrical Impedance Spectroscopy in Non-melanoma Skin Cancer. Acta Dermato-Venereologica, 100(18), (2020), 1-5; U.S. Pat. No. 9,636,035).

In addition, untreated intraocular tumors, including uveal melanoma and pediatric retinoblastoma, result in vision loss and are associated with high mortality rates. The metastatic cases may have a survival rate as low as 50%. These tumors may also cause retinal detachment, secondary glaucoma, and complete vision loss. To preserve vision and improve survival rate, it is important to detect the tumors in the early stage of development and act accordingly. However, patients with choroidal and ciliary body melanoma tend to be asymptomatic. In addition, ciliary body melanoma is much more difficult to visualize because of the anatomical location.

The detection of intraocular tumors, including uveal melanoma and pediatric retinoblastoma, also presents special problems. For example, melanoma may grow in different parts of the uveal tract, such as the iris, ciliary body, and choroid. Tumors in the ciliary body and choroid are more difficult to detect and often require dilation and/or specialized ophthalmic ultrasound. Left untreated, these tumors can result in vision loss and are associated with a high mortality rate. Thus, early diagnosis and timely treatment while tumors are small is critical in reducing the risk of metastasis and improving survival rate. But because these tumors can go unnoticed by patients, they can remain undiscovered until presented to a doctor.

An unmet need exists for a truly non-invasive, easily accessible, low-cost, and patient-driven way to identify/detect conditions such as cutaneous melanoma and intraocular tumors.

### SUMMARY

In an embodiment, a patch, for measuring electrical impedance in biological tissue comprises a flexible substrate, a resonator circuit on the flexible substrate comprising an inductor and a capacitor; and first and second electrical contacts electrically connected to the resonator, which are exposed on a surface of the patch, for making electrical contact with the biological tissue.

In another embodiment, a system for measuring impedance in biological tissue comprises (a) a patch comprising a flexible substrate, a first resonator circuit on the flexible substrate comprising a first inductor and a first capacitor electrically coupled in parallel, and first and second electrical contacts electrically connected in parallel with the resonator circuit, which are exposed on a surface of the patch, for making electrical contact with the biological tissue and (b) a reader comprising a second resonator circuit comprising a second inductor and a second capacitor electrically coupled in parallel, an electric oscillator coupled to provide for periodic electrical signal across the second resonator circuit, and a voltage monitoring circuit coupled to read a voltage across the second resonator, wherein the reader is configured such that the first inductor will inductively couple with the second inductor to transfer energy from the oscillator to the first resonator circuit via the inductive coupling when the reader is positioned in proximity to the patch.

In yet another embodiment, a method of detecting a biological condition of biological tissue comprises providing a patch comprising a flexible substrate, a first resonator circuit on the flexible substrate comprising a first inductor and a first capacitor electrically coupled in parallel, and first and second electrical contacts electrically connected in parallel with the resonator circuit, which are exposed on a surface of the patch, for making electrical contact with the biological tissue, providing a reader comprising a second resonator circuit comprising a second inductor and a second capacitor electrically coupled in parallel, an electric oscillator coupled to provide for periodic electrical signal across the second resonator circuit, and a voltage monitoring circuit coupled to read a voltage across the second resonator, disposing the patch such that the first and second electrical contacts are in electrical contact with a first biological tissue, positioning the reader in proximity of the patch such that the first inductor and the second inductor inductively couple, applying a periodic signal across the second resonator circuit, measuring a first voltage across the second resonator, and evaluating the first measured voltage to determine a biological condition of the first biological tissue.

### BRIEF DESCRIPTION OF THE DRAWINGS

A more detailed understanding may be had from the detailed description below, given by way of example in conjunction with the drawings appended hereto. Figures in such drawings, like the detailed description, are exemplary. As such, the Figures and the detailed description are not to be considered limiting, and other equally effective examples are possible and likely. Furthermore, like reference numerals (“ref.”) in the Figures (“FIGs.”) indicate like elements, and wherein:

FIG. 1 is a circuit diagram showing the components of the system in accordance with embodiments;

FIG. 2 is a graph of the voltage of the reflected signal as a function of frequency for a test signal under two different conditions;

FIGS. 3A, 3B, 3C, and 3D are graphs of voltage of the reflected signal as a function of time illustrating examples of possible effects of diseased tissue on the reflected electrical signal as compared healthy tissue;

FIG. 4A is an elevation view of the layers of a patch in accordance with some embodiments;

FIGS. 4B and 4C are top and bottom views, respectively, of the circuit-bearing layer of a patch in accordance with some embodiments;

FIG. 4D shows the patch disposed on a hand in accordance with some embodiments;

FIG. 4E is a diagram illustrating the layers and electrical components of a smart, chipless, battery-free contact lens (SCBC) in accordance with some embodiments;

FIG. 5 illustrates how an SCBC may be placed on an eyeball in various orientation to obtain electric impedance data for locating a tumor in the eye;

FIG. 6A illustrates the components in an eyeball that have a significant effect on the electrical impedance of the eyeball; and

FIG. 6B illustrates equivalent electric circuit model for the elements of FIG. 6A.

### DETAILED DESCRIPTION

In the following detailed description, numerous specific details are set forth to provide a thorough understanding of embodiments and/or examples disclosed herein. However, it will be understood that such embodiments and examples may be practiced without some or all of the specific details set forth herein. In other instances, well-known methods, procedures, components, and circuits have not been described in detail, so as not to obscure the following description. Further, embodiments and examples not specifically described herein may be practiced in lieu of, or in combination with, the embodiments and other examples described, disclosed or otherwise provided explicitly, implicitly, and/or inherently (collectively “provided”) herein.

In accordance with embodiments, medical/biological conditions may be detected by use of a noninvasive apparatus that detects the electrical properties of bodily tissue and compares the properties of healthy tissue to potentially unhealthy tissue to detect abnormalities that are potential health risks. More particularly, embodiments may be utilized for diagnosing/detecting/monitoring a diseased condition of the skin, eye, mucous membranes, etc., of a subject, particularly the presence of skin cancer, e.g. basal cell carcinoma or malignant melanoma, a squamous cell carcinoma or precursors thereof, and the presence of intraocular tumors, including uveal melanoma and pediatric retinoblastoma, using impedance measurements.

In accordance with embodiments, biological conditions that are precursors of skin cancer, such as, actinic keratoses (a precursor of squamous cell carcinoma) and dysplastic nevi (a precursor of malignant melanoma), may be diagnosed and/or detected using the apparatus and methods described herein.

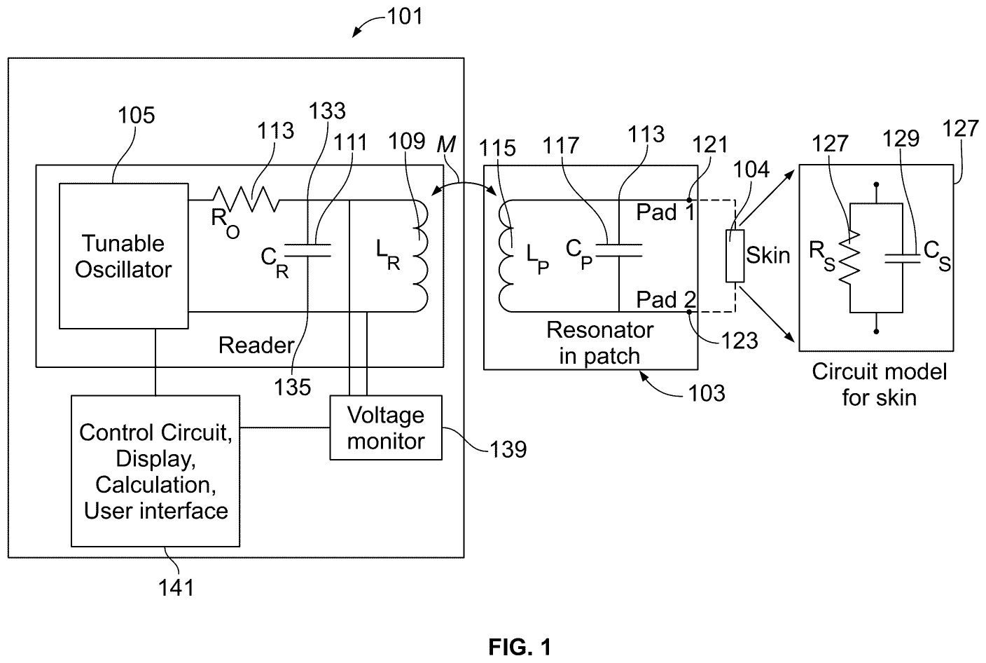

With reference to FIG. 1, an apparatus comprising a base unit 101 (sometimes referred to herein as a reader or reader unit) inductively couples (i.e., wirelessly through the air) to a probe unit 103 (sometimes referred to herein as a patch) that is placed in contact with the bodily tissue under investigation 104. The reader unit 101 comprises an electrical oscillator circuit 105 that generates an electrical impulse signal, such as a sinusoidal waveform (e.g., continuous or pulsed) that passes through a parallel LC circuit (e.g., comprising an inductor 109 and a capacitor 111) or an RLC circuit (comprising a resistor 113 placed in series between one terminal of the oscillator 105 and the LC circuit).

The patch unit 103 comprises another LC circuit comprising another parallel coupled inductor 115 and capacitor 117 and a pair of electric terminals (electrodes) 121, 123 coupled in parallel with the LC circuit, which electrodes 121, 123 can be placed in contact with the tissue under investigation. A parallel LC circuit, such as the one formed by capacitor 111 and inductor 109 or the one formed by capacitor 117 and inductor 115, essentially is an electrical resonator and may sometimes be referred to herein as a resonator or resonator circuit. In some embodiments, a resistor may also be included in the patch circuitry in series with the resonator. The patch circuitry is disposed directly on a flexible membrane that can be secured to tissue such that the two electrodes 121, 123 are in contact with the tissue.

In operation, the reader unit 101 is brought into close proximity with the patch 103 so that the inductor 109 of the reader unit inductively couples (represented by inductive coupling force M in FIG. 1) with the inductor 115 of the patch 103 so as to provide the electrical impulse signal to the LC circuit of the patch and to any tissue to which the electrodes 121, 123 are attached. The tissue that is positioned between the two electrodes 121, 123 may be electrically modelled in many ways. In one such as shown in FIG. 1, it may be modelled as a parallel RC circuit comprising a resistor 127 and a capacitor 129 coupled in parallel.

The electrical circuitry on the patch side of the inductive coupling M will cause a reflectance through the inductive coupling, M, that will have an effect on the voltage across the parallel LC circuit of the reader 101 (e.g., the voltage across nodes 133 and 135), which effect is a direct result of the impedance of the circuitry on the patch side of the inductive coupling, including the impedance of the tissue that the two electrodes 121, 123 are in contact with. Since the values of the circuit components built into the patch (i.e., inductor 115 and capacitor 117) and the circuit components built into the reader (i.e., inductor 107, capacitor 109 and resistor 111) are all fixed and known, and, therefore, can be accounted for mathematically, the voltage across nodes 133 and 135 can be used as a measurement of the impedance of the tissue across which electrodes 133 and 135 are coupled.

In turn, the impedance of that tissue can be used as an indicator of an abnormal medical/biological condition of that tissue. Thus, in an embodiment, the reader unit 101 further includes a circuit 139 that detects the voltage across nodes 133 and 135. In an embodiment, circuit 139 may be an LCR meter.

The reader unit also includes circuitry 141 for controlling the reader in accordance with the descriptions herein, including circuitry for controlling the oscillator to generate the signals described herein, processing circuitry for performing any calculations described herein and running diagnostics of the reader unit itself, interface circuitry such as a display screen for displaying relevant information (such as the voltages measured by the voltage monitor 139, on/off status of the device, calculated impedances of the tissue, etc.), user interface equipment (such as buttons for turning the unit on and off, initiating a measurement, initiating diagnostics of the device, etc.).

In general, the impedance of the tissue will affect one or both of the amplitude of the sinusoidal waveform detected across nodes 133 and 135 and the phase of the sinusoidal waveform across nodes 133 and 135. The electrical resistance and capacitance of the tissue causes changes in the amplitude and phase of the reflected signal. The tissue may also affect the frequency and/or quality factor of the patch. Thus, the amplitude, phase, quality factor, and/or frequency of the reflected signal measured across nodes 133 and 135 may be used as an indication of a medical condition of the tissue between the electrodes 121, 123.

Depending on the robustness of the available data as to the particular effect of a particular medical/biological condition on the impedance of tissue, it may be possible to detect the presence of a medical/biological condition of tissue based solely on measurement of the impedance of that tissue. However, in other cases, it is more useful to measure the delta (difference) between the impedance of the tissue under investigation and known (or assumed) healthy similar tissue to detect a significant difference therebetween. Particularly, since there are many uncontrolled factors that typically factor into the impedance of any particular tissue, it is advisable to compare the impedance of the tissue under investigation with similar healthy tissue from the same patient to minimize the impact of such uncontrollable factors. Tumors, for instance, typically have a lower impedance than healthy tissue.

More particularly, in an embodiment, the difference between the impedance of the tissue under investigation and similar healthy tissue from the same body may be used as an indicator of a biological condition of the tissue under investigation. Let us consider the example of use of the apparatus for detection of uveal tumors in the eye. The presence of a uveal tumor in an eyeball will cause the eyeball to have a different overall impedance that if no tumor was present in the eyeball. Statistically, it is extremely unlikely that a person (or other animal) will have a uveal tumor in both eyes. Accordingly, comparing the impedance readings of one eye of an individual against the impedance readings of the other eye of the same individual will usually lead to a proper detection of a medical condition in one of the eyes.

Thus, in one simple embodiment, the detection of a difference (or delta) in one or both of the voltage amplitude detected across nodes 133 and 135 and/or the phase of the signal across node 133 and 135 when measured in each eye exceeding a certain threshold can be used as an indicator of a medical/biological condition in one of those eyes.

In some embodiments, the apparatus may be used as a preliminary indication of a potential medical/biological condition indicating nothing more than a need for further, more intrusive or more robust analysis. For instance, in one embodiment, the apparatus may be implemented as a low cost, home kit for individuals to determine if they should see a doctor about a new mole.

Depending on the robustness of the available data as to how a particular medical condition affects the impedance of a particular tissue, in some embodiments, the difference between the measurements at the two sites might not even indicate which of the two sites is the potentially unhealthy site, but only that there is a significant, unexpected difference between the two eyes (indicating that one of the eyes is probably subject to some unusual medical/biological condition).

If, on the other hand, the data is more robust (e.g., it is know that the presence of a uveal tumor generally causes the voltage amplitude of the reflected signal to be lower than in an eye without a uveal tumor), then it would be possible to predict that the eye with the lower amplitude is the potentially unhealthy eye.

Again depending on the robustness of the available data as to how different conditions affect the impedance of particular tissues, it may or may not be reasonable to make an actual diagnosis as to the particular potential medical/biological condition of the tissue under investigation. For example, hypothetically, it may be known that first medical condition, e.g., a uveal tumor, may cause the resistance of the skin to increase (such that the voltage detected across nodes 133 and 135 will decrease) while the capacitance decreases (such that the phase of the reflected signal across nodes 133 and 135 will lag the phase of the input signal by the oscillator), whereas, hypothetically, a different condition, e.g., a retinal blastoma, generally causes the resistance of the tissue to decrease (such that the voltage detected across nodes 133 and 135 will increase) and the capacitance of the tissue decrease (such that the phase of the reflected signal across nodes 133 and 135 will lag the phase of the input signal by the oscillator). If that is known, then the difference in readings between the healthy site and the site under investigation may be useful in diagnosing the actual medical condition (e.g., uveal tumor versus retinal blastoma).

Since the equipment needed to implement the apparatus, techniques and methods disclosed herein can be manufactured extremely inexpensively (as compared to other medical devices and techniques for detecting tumors, etc.), it is envisioned In one embodiment as being a low-cost, over-the counter product used by patients for self-diagnosis of one or more potentially unhealthy conditions. In such cases, the device may be used only as a preliminary determination that the patient should see a doctor for a more robust evaluation of the potentially condition(s).

The nature of the expected difference in impedance between healthy tissue and unhealthy similar tissue caused by any particular medical/biological condition can be determined in many ways. For instance, data may be collected empirically or experimentally over many patients and years. Alternately or additionally, it may be possible to determine likely differences in impedance between healthy tissue and diseased tissue by calculation based on the known difference in the electrical properties of different types of tissues. Regardless of the particular technique for determining the expected impedance delta caused by the particular medical/biological condition, if the data is sufficiently robust, it can be used to convert the measured impedance delta into a diagnosis of a likely biological condition.

Depending on the medical condition being screened for, in some cases, it may be that the delta in only the phase of the reflected signal or the delta in only the amplitude of the reflected signal or the delta in only frequency of the reflected signal may be sufficient to make a diagnosis (or at least indicate the need for further medical intervention). In other cases, it may be a combination of any two or more of the phase, amplitude, quality factor, and frequency of the reflected signal. In yet other cases, a more detailed analysis of the reflected signal may be advisable, such as calculating the actual resistance and/or capacitance of the tissue from the phase, frequency and/or amplitude of the reflected signal.

The reader unit may be programmed to perform any of the calculations or operations necessary to make any such determinations and to display relevant diagnosis information to the user.

In one embodiment, the oscillator may be operated so as to output a sinusoidal signal. In some embodiments, the sinusoidal signal may be continuous. In other embodiments, the sinusoidal signal may be pulsed (e.g., turned on and off at regular intervals). An advantage of pulsing the sinusoidal signal is that it permits better measurement of noise in the reflected signal. Particularly, in one exemplary embodiment, measurements may be taken of the signal across nodes 133 and 135 during the periods when there is no input signal from the oscillator (i.e., during the off portion of the duty cycle of the oscillator signal) and any signal detected can be considered noise. Then, the noise can be subtracted from the signal readings during the on portion of the duty cycle to give a more accurate reading of the impedance of the tissue.

In one exemplary embodiment for detecting cutaneous melanoma (skin cancer), the patch may be placed on the surface of the skin such that contact electrodes 121 and 123 are in electrical contact with the surface of the eye on opposite sides of a suspicious mole. The reader unit is brought within close proximity of the patch so as to cause inductor 109 on the reader to magnetically couple with inductor 115 on the patch so as to create the inductively coupled circuit as shown in FIG. 1. In one preferred embodiment, the inductors 111 and 115 are formed as flat windings disposed on a printed circuit board substrate and a flexible substrate, respectively. Thus, for best magnetic coupling, the two substrates/inductors should be oriented parallel to each other and as close as practical to each other. For instance, the inductor 111 in the reader unit may be disposed parallel and close to a flat surface of the reader unit 101 so that the reader unit may be placed in flat contact with the patch to minimize the distance between the two inductors and to keep the two inductors parallel to each other. Preferably, the inductors are positioned parallel to each other and less than 10 mm from each other, more preferably, 4 mm or less apart, and, most preferably, 2 mm or less apart.

The oscillator is then controlled to output a sinusoidal signal at a plurality of frequencies surrounding the resonance frequency of the unloaded resonators, in this case, between about 4 Mhz and 5 Mhz at an amplitude of 1 Volt. The optimal oscillator frequency range to use in any given embodiment can be almost anything and depends on the value of the selected capacitors and inductors for the two resonator circuits. The values may be selected based on many factors, including the desire to keep the circuit elements as small as practical and to keep the cost of the oscillator as low as reasonable. A good compromise is to select small values that cause the resonant frequency of the two resonator circuits in the absence of a load across the electrodes to be (a) the same for both resonators and (b) in the range of 0.1-20 MHz. then select a range around that resonant frequency that is large enough to assure that any shifted resonance frequency of the resonators due to the tissue positioned between the electrodes will be within that range. In this example, values that yielded an unloaded resonance frequency of about 4.5 Mhz were chosen. Since it is extremely unlikely that the load of any tissue that might be placed across the electrodes will cause the resonance frequency to shift by more than about 10% at these frequencies, measuring for resonance frequency with a load across the electrodes over a frequency spread of about 1 MHz around the unloaded resonance frequency (i.e., 500 KHz in either direction, namely 4-5 MHz) should be more than adequate to encompass any shift in the resonance frequency of the resonators when the patch is attached to tissue. At lower frequencies, the possible frequency shift may increase as a function of the unloaded resonance frequency, e.g., to 20% or more). Thus, at lower frequencies the range may be increased accordingly.

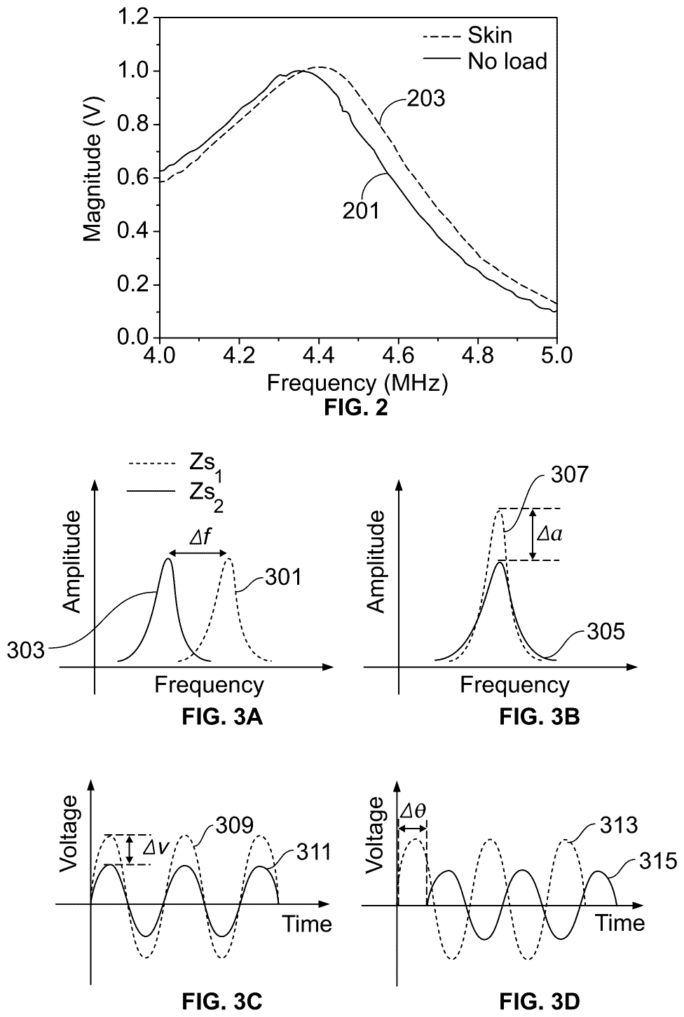

The reflected signal across nodes 133 and 135 is read at each such frequency to determine the resonant frequency of the overall circuit. With reference to FIG. 2, the resonant frequency is the frequency at which the peak to peak amplitude of the measured sinusoidal signal across electrodes 133 and 135 is at its maximum. For purposes of illustration, FIG. 2 shows two such peak voltage curves, namely, (1) curve 201, which is the curve when the patch is not in contact with any tissue such that the circuit comprises the components shown in the patch and the reader with pad1 and pad 2 open circuited (the patch is not in contact with any tissue) and (2) curve 203, which is the curve when the patch is in contact with the skin of a patient such that the circuit comprises the components shown in the patch and the reader with pads 121 and 123 coupled across a section of the patient's skin such that the impedance of the patient's tissue between the two electrodes 133, 135 forms part of the circuit (in parallel with the inductor 115 and capacitor 117).

As can be seen, the presence of the skin shifts the resonant frequency from about 4.33 MHz to about 4.41 MHz.

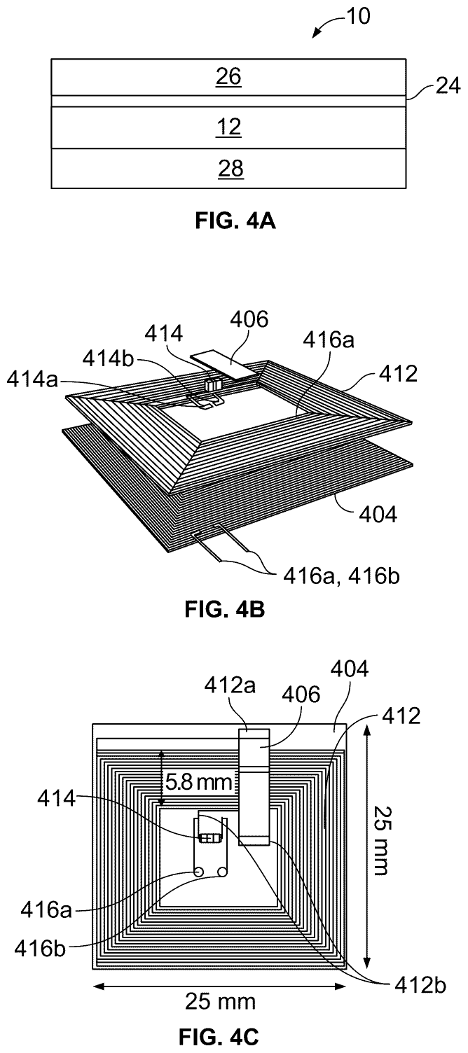

Referring now to FIGS. 3A-3D, they are graphs showing four possible ways in which a difference in electrical impedance of diseased tissue versus healthy tissue may be manifested in the reflected signal. FIGS. 3A-3D are for illustrative purposes and do not represent any actual measurements. For instance, FIGS. 3A-3C illustrate different effects individually (isolated from any of the other effects). However, in a real-life scenario, it is likely that the two or more of these effects will exist simultaneously in the reflected signal.

Referring first to FIG. 3A, it shows plots of the magnitude of the reflected sinusoidal signal as a function of frequency. It reveals the resonance frequency of the circuit (i.e., the frequency at which the magnitude is the greatest). Plot 301 shows the plot for when the patch is placed over healthy tissue and plot 303 shows the plot for when the patch is placed over diseased tissue. As can be seen, the diseased tissue causes the resonant frequency to shift downwardly (which means that the capacitance of the diseased tissue is higher than the capacitance of the healthy tissue).

FIG. 3B is another plot of the magnitude of the reflected sinusoidal signal as a function of frequency. Plot 305 shows the plot for when the patch is placed over healthy tissue and plot 307 shows the plot for when the patch is placed over diseased tissue. As can be seen, in this case, the diseased tissue causes the magnitude of the reflected signal to decrease (meaning that the overall impedance of the diseased tissue is less than the impedance of the healthy tissue), but does not alter the resonant frequency.

FIG. 3C shows plots of the voltage of the reflected sinusoidal signal as a function of time at a given frequency. Plot 309 shows the plot for when the patch is placed over healthy tissue and plot 311 shows the plot for when the patch is placed over diseased tissue. As can be seen, in this case, the diseased tissue causes the voltage of the reflected signal to decrease, but does not alter the frequency or phase of the signal. FIG. 3C reveals information very similar to the information revealed by FIG. 3B (that the overall impedance of the diseased tissue is less than the impedance of the healthy tissue).

Finally, FIG. 3D is another plot of the voltage of the reflected sinusoidal signal as a function of time. Plot 313 shows the plot for when the patch is placed over healthy tissue and plot 315 shows the plot for when the patch is placed over diseased tissue. As can be seen, in this case, the diseased tissue causes changes in both the phase and the amplitude of the reflected signal relative to the reflected signal for healthy tissue, but does not cause any change in the frequency of the reflected signal.

In certain embodiments the apparatus and methods described herein may be used for the detection of various cancers in mammals, particularly humans, in or on the skin, in or on the eye, in or on mucous membranes in the buccal and nasal cavities, etc., and including all other parts of the body, such as the breasts.

In all aspects of the invention described herein, the description applies to and includes the use on mammals generally, with particular emphasis on humans.

In preferred embodiments, the reader includes a display device, such as an LCD or LED screen capable of providing/displaying total impedance observed and/or the magnitude of the voltage across the reader resonator, when brought into magnetic coupling with the patch. In some embodiments, it may display the delta between two successive measurements. In yet other embodiments, it may display a diagnosis or other recommendation based on a measurement (or the delta between two successive measurements). The reader unit may provide control interface features to allow the user to select what information he/she would like displayed in response to one or more measurements.

In one embodiment, the apparatus provides a method of detecting cancer by applying the patch to an area of the body of interest, bringing the reader into magnetic coupling with the patch, and obtaining values for at least one of total impedance observed and the magnitude of the voltage across the reader resonator, followed by optionally comparing the impedance and/or voltage values obtained with known and/or comparative values in order to determine the presence or absence of cancer.

According to a further aspect, there is provided a method for measuring and/or monitoring and/or detecting biological conditions of a subject over time, for example, changes in skin properties of a subject, or changes in tissue properties of a subject, by the taking of repeated impedance and/or voltage measurements over the course of time and cataloguing and comparing the values.

This invention provides several advantages. One advantage is that impedance/voltage phenomena that manifest at the surface of the stratum corneum, at the surface of the eye, at the surface of the mucous membranes, etc., can be assessed in a wholly noninvasive and reliable manner without disturbance, penetration, ingress, or irritation of the surface.

Another advantage is that the patch 103 according to the present invention need only be held against the surface being measured for a short period of time and with no need for substantial applied pressure. For example, for skin, the patch can simply be contacted to the skin and held there with only enough pressure to maintain contact, for example with the hand or fingers, or with medical tape. For the eye, when the patch is in the form of a contact lens, no external applied pressure may be required. Thereby, as the devices are less dependent on the applied pressure, they are less operator dependent. This advantage also entails significantly less inconvenience for the patient or person subjected to testing.

The reader unit 101 may be constructed in accordance with well-known principles in the art of electronic circuits. For instance, it may comprise one or more computer processors for performing the operations described herein such as measuring the voltage across the nodes 133 and 135 as represented by block 139 in FIG. 1 and performing all of the functions and operations described herein for block 141 in FIG. 1, including, for instance, controlling a display device, performing any calculations described herein and providing a user interface for operating the device.

In a preferred embodiment, the patch contains only an inductor, a capacitor and the electrodes as shown in FIG. 1. Such circuitry can be fabricated on a flexible substrate.

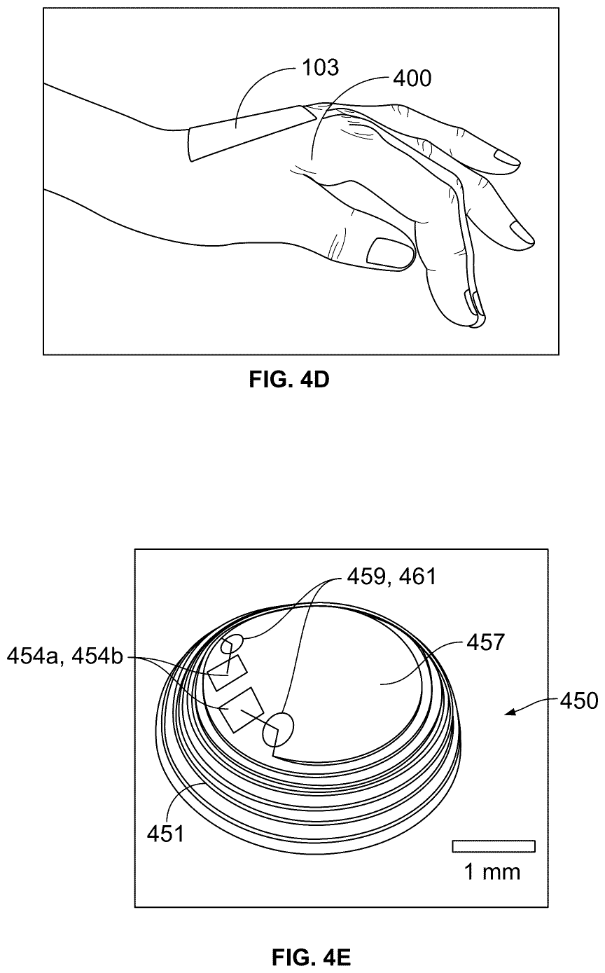

FIG. 4A is a side view of an exemplary patch 103 for use in contacting the skin of a patient (such as in connection with embodiments in which the apparatus is used examine moles or other skin features for cancer) showing the various layers thereof in accordance with embodiments. In this aspect, all the circuit components are present in one layer, however, in different embodiments such as will be described below, the components may be present in different layers or embedded within the substrate.

As show in FIG. 4A, a component layer 24 containing the circuitry (i.e., the inductor and capacitor) is on a flexible insulating substrate 12. These two layers are sandwiched between an optional flexible insulating top layer 26 and an optional flexible insulating bottom layer 28. Electrical contacts, for contacting tissue, will extend from the component layer 24 through the substrate of the component layer and through the optional flexible insulating bottom layer 28 so that they may be made to contact the tissue under investigation by placing the patch in contact with the skin (with or without adhesive).

The substrate, optional top layer, and bottom layer may be formed from any flexible insulating polymer or plastic. The term “flexible” as used herein refers to the ability to bend without breaking. In certain embodiments the flexible substrate has a modulus of elasticity E of about 100,000-500,000 psi. In certain embodiments the flexible substrate has an effective Young's modulus less than or equal to 10 GPa, less than or equal to 5 MPa, or optionally less than or equal to 1 MPa and optionally for some applications less than or equal to 0.1 MPa.

Preferably, the polymer material is biocompatible, such as a silicone elastomer (for example, polydimethylsiloxane (PDMS)), poly(butylene adipate-co-terephthalate) (PBAT) (such as ECOFLEX®), polylactic acid, polyimide, and blends and copolymers thereof. Hydrogels and other biocompatible gels may be used, so long as the component layer is sealed or otherwise protected from moisture. Examples include polyacrylamide, polyvinyl pyrrolidone (PVP), silicone hydrogels, polyurethanes (such as thermoplastic polyurethanes) and hydrogels used in contact lenses (for example tefilcon, hioxyfilcon A, lidofilcon, omafilcon A, hefilcon C, phemfilcon, methafilcon A and ocufilcon D) and mixtures thereof. Other examples include polymers and co-polymers of 2-hydroxyethylmethacrylate, glycerol methacrylate, methyl methacrylate, N-vinyl pyrrolidone, N-vinyl-2-pyrrolidone, 2-methacryloyloxyethyl phosphorylcholine, ethoxyethyl methacrylate and methacrylic acid. The hydrogel will also contain water, and may contain one or more salts such as sodium chloride, buffers, preservatives, plasticisers and polyethylene glycol. Examples of hydrogel thermoplastic polyurethanes (TPUs) include TECOPHILIC® thermoplastic polyurethanes. These TPUs offer an aliphatic, hydrophilic polyether-based resin which has been specially formulated to absorb equilibrium water contents from 20 to 1000% of the weight of dry resin. Examples of TPUs include TECOPHILIC® SP-80A-150 (“SP-80A-150”) and TECOPHILIC® Hydrogel TG-500 (TG-500”), manufactured by LUBRIZOL®. Silicones consist of an inorganic silicon-oxygen backbone chain with organic side groups attached to the silicon atoms. Silicones have in general the chemical formula [R2SiO]n, where R is an organic group such as an alkyl or phenyl group. Other polymers which may be used include medical-grade polymers approved for body contact. Examples of suitable plastics and polymers include acetal copolymer, acetal homopolymer, polyethylene terphthalate polyester, polytetrafluoroethylene, ethylene-chlorotrifluoro-ethylene, polybutylene terephthalate-polyester, polyvinylidene fluoride, polyphenylene oxide, polyetheretherketone, polycarbonate, polyethylenes, polypropylene homopolymer, polyphenylsulfone, polysulfone, polyethersulfone, and polyarylethersulfone. If the polymer used for the substrate of layer 24 is not biocompatible, then a biocompatible polymer should be used as a bottom layer 28. The layers may be adhered to each other using heat to fuse the edges, co-extrusion or co-injection, interlocking mechanical connections, encapsulation and/or with an adhesive, including a biocompatible sealant such as LOCTITE® medical device adhesive. Rigid materials having a very low thickness so they are sufficiently flexible, such as silicon, may also be included. The composition of each of the substrate 24, optional top layer 26, and bottom layer 28 may be chosen independently.

One group of plastics useful for the flexible substrate 24 include polyimide, heat stabilized polyethylene terephthalate (HS-PET), polyethylenenapthalate (PEN), polycarbonate (PC), polyarylate (PAR), polyetherimide (PEI), polyethersulphone (PES), polyimide (PI), Teflon® poly(perfluoro-alboxy) fluoropolymer (PFA), poly(ether ether ketone) (PEEK), poly(ether ketone) (PEK), poly(ethylene tetrafluoroethylene) fluoropolymer (PETFE), poly(methyl methacrylate), acrylate/methacrylate copolymers (PMMA), cyclic polyolefins, ethylene-chlorotrifluoro ethylene (E-CTFE), ethylene-tetra-fluoroethylene (E-TFE), poly-tetrafluoro-ethylene (PTFE), fiber glass enhanced plastic (FEP), high density polyethylene (HDPE).

Another group of plastics useful for the flexible substrate 24 include polyimide, polyethylene terephthalate (PET), polyurethane, acrylates, acetal polymers, biodegradable polymers, cellulosic polymers, fluoropolymers, nylons, polyacrylonitrile polymers, polyamide-imide polymers, polyarylates, polybenzimidazole, polybutylene, polycarbonate, polyesters, polyetherimide, polyethylene, polyethylene copolymers and modified polyethylenes, polyketones, poly(methyl methacrylate), polymethylpentene, polyphenylene oxides and polyphenylene sulfides, polyphthalamide, polypropylene, styrenic resins, sulphone based resins, and vinyl-based resins.

Another group of plastics useful for the flexible substrate 24 include low density polyethylene (LDPE), high density polyethylene (HDPE), polypropylene (PP), polyvinyl chloride (PVC), polystyrene (PS), nylon, Teflon, and thermoplastic polyurethane (TPU).

Another group of plastics useful for the flexible substrate include acetal polymers, biodegradable polymers, cellulosic polymers, fluoropolymers, nylons, polyacrylonitrile polymers, polyamide-imide polymers, polyimides, polyarylates, polybenzimidazole, polybutylene, polycarbonate, polyesters, polyetherimide, polyethylene, polyethylene copolymers and modified polyethylenes, polyketones, poly(methyl methacrylate), polymethylpentene, polyphenylene oxides and polyphenylene sulfides, polyphthalamide, polypropylene, polyurethanes, styrenic resins, sulfone based resins, vinyl-based resins, rubber (including natural rubber, styrene-butadiene, polybutadiene, neoprene, ethylene-propylene, butyl, nitrile, silicones), acrylic, nylon, polycarbonate, polyester, polyethylene, polypropylene, polystyrene, polyvinyl chloride, and polyolefin.

Another group of plastics useful for the flexible substrate 24 include thermoplastic elastomers, styrenic materials, olefenic materials, polyolefin, polyurethane thermoplastic elastomers, polyamides, synthetic rubbers, PDMS, polybutadiene, polyisobutylene, poly(styrene-butadiene-styrene), polyurethanes, polychloroprene, silicones, polysiloxanes including poly(dimethyl siloxane) (i.e. PDMS and h-PDMS), poly(methyl siloxane), partially alkylated poly(methyl siloxane), poly(alkyl methyl siloxane) and poly(phenyl methyl siloxane), silicon modified elastomers, thermoplastic elastomers, styrenic materials, olefenic materials, polyolefin, polyurethane thermoplastic elastomers, polyamides, synthetic rubbers, polyisobutylene, poly(styrene-butadiene-styrene), polyurethanes, polychloroprene and silicones. Another group of plastics useful for the flexible substrate 24, particularly when the

patch is in the form of a contact lens, includes all those plastics discussed above and further includes PMMA, polymer hydrogels including hydroxy ethyl methacrylate (HEMA) hydrogels, silicone hydrogels, PVA, PVA hydrogels, PEG, RGP, NVP, EGDMA, PDMS, PDMS, DA—diacetone acrylamide; DMA—N,N-dimethylacrylamide; HEMA—2-Hydroxyethyl methacrylate; MAA—methacrylic acid; MMA—methyl methacrylate; NCVE-N-carboxl vinyl ester; NVP—N-vinyl pyrrolidone; PBVC—poly[dimethylsiloxyl] di[silybutanol] bis[vinyl carbamate]; PC—phosphorylcholine; TPVC—tris-(trimethylsiloxysilyl) propylvinyl carbamate; and TRIS—tris-(hydroxylmethyl) aminomethane.

Preferably, each of the substrate 24, optional top layer 26, and optional bottom layer 28 may independently have a thickness of 5 to 500 μm, more preferably 25 to 200 μm, including 30, 40, 50, 60, 70, 80, 90, 95, 100, 110, 120, 130, 140, 150, 160, 170, 180 and 190 μm. Preferably, the layers have a length and width sufficient to contain all the desired components of the component layer, and has a size sufficient for an adult to grasp and place on the skin by hand. Preferably the substrate, optional top layer and optional bottom layer have a width of 0.25 to 15 cm, more preferably 0.5 to 10 cm, including 0.75, 1, 1.25, 1.5, 1.75, 2, 2.25, 2.5, 2.75, 3, 3.5, 4, 4.5, 5, 6, 7, 8 and 9 cm. Preferably the substrate, optional top layer and optional bottom layer have a length of 0.25 to 15 cm, more preferably 0.5 to 10 cm, including 0.75, 1, 1.25, 1.5, 1.75, 2, 2.25, 2.5, 2.75, 3, 3.5, 4, 4.5, 5, 6, 7, 8 and 9 cm. Alternatively, thicknesses can range from, e.g., 0.1-30 mm, including for example 0.5, 1, 2, 4, 6, 8, 10, 12, 14, 16, 18 and 20 mm. Depending on its shape, and to the extent the flexible substrate can be characterized by a length and width, or a diameter, typical values for each include 5-100 mm, and may be up to 400-500 mm and more depending on the application envisioned. The device may have any shape, including rectangular, circular, oval, 2-lobed, 3-lobed, 4-lobed, an irregularly shaped. When the resonator is used for the eye, the dimensions and shape should be the same as used from contact lenses, although it may also be larger to extend coverage to other portions of the eye.

Optionally, a biocompatible adhesive may be used on the underside of the substrate 24 or the optional bottom layer 28 for adhering the resonator to skin. Such an adhesive may not be necessary if the weight of the device is low enough and a polymer is used for the substrate or the optional bottom layer, that naturally sticks to skin without an adhesive, such as by fluid capillary forces, van der Waals forces, or other adhesion mechanisms. For example, when PDMS is used and the resonator has a weight of at most 120 mg, it may naturally stick to skin. In such a circumstance, an adhesive force of only 1-2 kPa is necessary to keep the device in place on skin during use. Examples of materials used for skin adhesion of a light weight device without an adhesive may be found in H. U. Chung et al. (“Binodal, wireless epidermal electronic systems with in-sensor analytics for neonatal intensive care,” Science, vol. 363, no. 6430, pp. 0-13, 2019).

Preferably, the resonator comprises a circuit comprising a capacitor and an inductor, which can be assembled or manufactured in any matter. For example, it is possible to combine electronic components and flexible substrates to form flexible (including extensible) electronic devices and equipment using various techniques such as the use of shadow masks and magnetron sputtering, mass printing of roll-to-roll system components, direct printing methods, 3D printing, stamping, nano-imprinting, lithography, etc. In one embodiment the patch 103 comprising a resonator on a flexible substrate can be made according to one or more of the disclosures in U.S. Published Patent Application No. 2020/0315488, B. S. Cook et al., “Inkjet-printed, vertically-integrated, high-performance inductors and transformers on flexible LCP substrate,” 2014 IEEE MTT-S International Microwave Symposium (IMS2014), Tampa, FL, USA, 2014, pp. 1-4, doi: 10.1109/MWSYM.2014.6848575, Y. Liu, et al., “Lab-on-Skin: A Review of Flexible and Stretchable Electronics for Wearable Health Monitoring,” ACS Nano, vol. 11, no. 10, pp. 9614-9635, 2017, M. Yuan et al., “Electronic Contact Lens: A Platform for Wireless Health Monitoring Applications,” Adv. Intell. Syst., vol. 2, no. 4, p. 1900190, 2020, H. U. Chung et al., “Binodal, wireless epidermal electronic systems with in-sensor analytics for neonatal intensive care,” Science (80-.)., vol. 363, no. 6430, pp. 0-13, 2019, Z. Ma, “An electronic second skin.,” Science, vol. 333, no. 6044, pp. 830-831, August 2011, and S. Niu et al., “A wireless body area sensor network based on stretchable passive tags,” Nat. Electron., vol. 2, no. 8, pp. 361-368, 2019, all incorporated herein by reference. The size, shape, and other characteristics of the pads, capacitor, and inductor are not limited.

Both the capacitor and inductor can be of any design, without limitation.

Preferably, the patch inductor is provided with at least two areas, for example, the electrodes 121, 123 thereof, that are exposed at a same surface of the patch and which are capable of making contact with another surface, for example a surface to which the patch is applied, such as human skin or the eye.

In a preferred embodiment the patch 103 is capable of conforming to the body—e.g., to the skin, the eye, the mucous membranes, etc. “Conforming” refers to a bending stiffness sufficiently low to allow the patch to adopt a desired contour profile, for example a contour profile allowing for conformal contact with a surface of skin, the eye, the mucous membranes, etc. while remaining functional.

When the flexible substrate is a contact lens it can be manufactured by any known method of manufacturing therefor and need not be of optical quality. See, e.g., U.S. Pat. Nos. 3,808,178, 4,152,508, 4,330,383, 4,216,303, 4,242,483, 4,248,989, 4,182,822, and 4,347,198, all incorporated herein by reference.

Preferably, the patch is not independently powered, has no battery, and is chipless. Preferably, the patch does not contain pins, needles or other sharp elements which could penetrate skin or a tissue surface.

In operation to take a reading in accordance with one particular exemplary embodiment, the patch is attached to the skin of a patient and the inductor 109 on the reader 101 is brought into close proximity (e.g., less than 10 mm, and preferably about 2-5 mm) to the inductor 113 of the patch 103 so that the inductors 109 and 113 magnetically couple without the need for any physical contact of the reader 101 with the patch 103. The oscillator 105 is turned on to inject the electrical input signal (e.g., a pulsed sinusoidal current that is varied from 4 MHz to 5 MHz over a short period of time, e.g., 1 second or less). Part of the current flows through the primary inductor 109 and the alternating magnetic flux from inductor 109 is coupled to the secondary inductor 113 on the patch with mutual inductance, M, and induces a corresponding alternating current in the patch circuitry. The voltage signal across nodes 133 and 135 is sensed by the voltage monitor circuit 139 on the primary side (reader), which signal includes the effects of the electrical properties of patient's skin. Since the values of all the electrical components of the reader and patch are known, it is possible to determine the impedance of the skin mathematically from the voltage readings across nodes 133 and 135. More particularly, with regard to the capacitance of the patient's skin, the resonance frequency of the circuit, ω₀, is given by:

$${ω}_{0} = \frac{1}{\sqrt{{L}_{p} ( {C}_{p} + {C}_{s} )}} \qquad ( 1 )$$

where Lₚ is the inductance of inductor 115, Cₚ is the capacitance of capacitor 117 and Cₛ is the capacitance of the skin. Thus, the resonance frequency, ω₀, can be determined from the readings of the voltage monitor 139, and, used in equation (1) to calculate the capacitance of the patient's skin, Cₛ.

Likewise, the resistance of the patient's skin, Rₛ, changes the total impedance observed from reader and affects the magnitude of the voltage across nodes 133 and 135 in the reader. The resistance of the skin is detected by the reader unit as a change in reflected impedance (Zr) according to:

$${Z}_{r} = \frac{{( ω M )}^{2}}{{z}_{pL}} \qquad ( 2 )$$

where Z_{pL} is the overall impedance of the patch including the load/tissue across the electrodes, ω is the frequency and M is the magnetic coupling coefficient. Thus, the shift in the voltage amplitude can be converted into the impedance of the skin.

In one embodiment, values of the both inductors 109 and 133 are 11.9 μHenry and the values of both capacitors 111 and 117 are 82 pFarads, thereby providing an unloaded resonance frequency of each resonator circuit of about 4.33 MHz.

The optimal specific values for the circuit elements, oscillator parameters, etc. will be highly dependent on various factors, such as the medical biological condition being tested for, the nature of the tissue to be tested, cost of components, the size of the tissue sample to be tested, etc. However, the following table shows some practical values for common scenarios.

FIGS. 4B and 4C are exploded and plan views, respectively, of the circuit layer (e.g., layer 24 in FIG. 4A) of an exemplary flexible patch 103, while FIG. 4D shows the complete patch 103 positioned on a hand 400. In an embodiment, the inductor is made of copper and is the square winding structure 412 with contacts at 412a and 412b. Electric tape 406 is conductive on one side and connects the two ends of the inductor coil to the two terminals 414a and 414b of the capacitor 414 so that it is coupled in parallel with the capacitor (as well as electrodes 416a and 416b).

TABLE 1

| Parameter | Value |
|---|---|
| Frequency range of oscillator input signal for testing | 10 kHz to 15 MHz |
| tissue | |
| Peak to peak voltage of the oscillator input signal | 100 mV-5 V |
| for testing tissue | |
| Pulse duration for a pulsed oscillator input signal | 20 mSec-500 mSec |
| Duty cycle for a pulsed oscillator input signal | 50% |
| Capacitor values | 82 pF for 5.09 MHz |
| Inductor values | 11.9 uH |
| Distance between reader and patch inductors during | 1 mm-10 mm |
| measurement | |

In an embodiment, the electrodes are copper and are seen at 416a and 416b. In an embodiment, the inductor and electrodes are implemented on a 100 μm thick polyimide 404 using a screen printing process and the size of polyimide layer is 25 mm×25 mm (but the overall patch (FIG. 4D) is 53 mm×44 mm). In an embodiment, a surface mounted capacitor seen at 414 with contacts 414a and 414b of size of 1 mm by 1 mm by 0.5 mm is attached to the substrate 404 to complete the resonator circuit.

For the reader unit 101 (not shown), the resonator circuit may constructed on a rigid substrate by soldering an external inductor and capacitor to a printed circuit board.

In an exemplary application, the reader was placed 4 mm away from the patch with the two inductors 109 and 115 oriented generally parallel to each other. A function generator applied the voltage in the reader unit at various frequencies from 4 MHz to 5 MHz. The resonant frequency of the device after applying a sinusoidal voltage of 1 Volt (peak-to-peak) with electrodes 133 and 135 open circuited (i.e., with the patch not contacting any skin) has its highest value at 4.33 MHz, whereas, after adding load (skin) by touching the hand of a 61-year-old female, the resonant frequency shifted to 4.41 MHz (note that the results discussed here are the results that are represented in FIG. 2).

Thus, the change in resonance frequency caused by the skin was about 80 kHz.

The change in voltage magnitude at the resonance frequency caused by the skin was 62 millivolts. That voltage delta increased to 656 millivolts at 4.6 MHz.

In the specific case of the eye, the apparatus, techniques, and methods disclosed herein may be used to detect unhealthy tissue in the eye, such as uveal tumors and retinoblastoma. More particularly, the electrical components described hereinabove for the patch, (i.e., inductor and capacitor in parallel, and two electrodes across the resonator) can be formed on a contact lens (hereinafter referred to as a smart, chipless, battery-free contact lens or SCBC) placed on the eye, and a reader unit such as previously described may be placed close to the SCBC to obtain measurements of the impedance parameters of the eyeball.

Uveal melanoma (UM) is the most common primary intraocular malignancy in adults, accounting for 85% of ocular cancers [3]. According to the Ocular Melanoma Foundation, approximately 2,500 individuals are diagnosed with UM in the United States annually. A mean age-adjusted incidence is 5.1 cases per million in the U.S. per year, while the annual incident rate in Scandinavia is 8 to 9 cases per million [4]. In the eye, both the primary tumor and the local treatment can negatively impact vision and eye health. Local treatment options include resection, radiation therapy and enucleation [5], [6]. Unfortunately, despite successful local treatment, UM is often a fatal disease due to the microscopic spread at the time of diagnosis. Posterior Ums arising in the choroid (90%) and ciliary body (6%) account for the majority of cases and are aggressive cancers with high risk for distant metastasis. Up to 50% spread to vital organs including the liver, lungs, and bones [7], [8], and there is no curative treatment for metastatic UM. The probability of death for patients with large (2:12 mm in basal diameter) metastatic UM tumors may be as high as 70% [7].

One strategy to preserve vision and improve the survival rate is early detection and treatment. The larger the tumor the greater the risk of metastasis [9]. In addition, local treatment options are more likely to be successful for smaller tumors [5]. Therefore, it is vital to detect and treat UM tumors when they are small.

The eyeball is nonhomogeneous and comprises various parts such as the cornea, anterior chamber, lens, vitreous humor, retina, uveal tract, and sclera. Each part has a different impedance and contributes to the overall impedance of the eyeball. These components are not electrically insulated and have relatively high conductivities in the range of 0.23-2 Sim at 13.5 MHz. The equivalent circuit of the eyeball, observed from the surface of the cornea, constitutes multiple capacitive-resistive circuits from different parts of the eye. If a tumor exists in any parts of the eye, it may change the electrical current density and voltage distribution in the eyeball and induce changes to the total impedance of the globe.

The impedance of both eyeballs may be measured, and abnormal differences in the impedance may be associated with the presence of a tumor. The healthy eyeball may be used as a reference, knowing that bilateral uveal melanoma is exceedingly rare (1 in 50 million) [2]; therefore, an abnormal difference in the impedance of a patient's two eyeballs will correlate to a risk of the existence of uveal melanoma.

Also, 75% of pediatric retinoblastoma (PR) is unilateral according to the American Cancer Society. Therefore, in most cases with PR, the other eye of the subject is healthy and can be used as a reference. In bilateral retinoblastoma, if the size, location, and stage of tumors are different on each eye; the unusual differences in the electrical impedance could be detectable and thus may still be an indication of retinoblastoma.

In use, a physician may place the SCBC on each eye (right and left) and measure the difference in the impedance of the two eyeballs. Although this will be a very effective method to detect unilateral cases, it can also be used to find bilateral dissimilar cases. In addition, in some embodiments, the SCBC may be manufactured as an over the counter screening test for individuals to measure the impedance of their eyeballs outside of the clinical setting and provide a tool for long-term, patient-driven, ongoing monitoring of the eyeballs.

Since it is believed that (1) melanoma induces change in the conductance and the permittivity of a uveal tract, and thus the existence of UM tumor changes the electrical impedance of the eyeball and (2) electrical impedance correlates with size, stage, and location of the lesion in the uveal tract, the SCBC may be used to measure the impedance of the eyes and identify abnormalities associated with UM.

With reference to FIG. 4E, which shows the structure and electrical circuits of the SCBC 450, in an embodiment, the SCBC comprises one inductor 451 and one capacitor 453 on a polydimethylsiloxane (PDMS) layer 455 to form a thin-film resonator on the contact lens 457. The contact lens itself may be a conventional contact lens, such as are used for correcting vision. The resonator will be electrically connected to the eyeball through contact electrodes 459, 461.

The impedance of the eyeball will change the parameters of the resonator, including one or more of resonance frequency, quality factor, amplitude, and/or phase angle of signals. The resonance frequency with electrodes 459, 461 open circuited (i.e., before the SCBC is placed on an eye) is given by:

$${f}_{r} = \frac{1}{2 π \sqrt{{L}_{s} {C}_{s}}} \qquad ( 3 )$$

The quality factor of the contact lens (Qs) depends on operation frequency, fₒ, inductance, Lₛ, and the resistance, Rₛ, of the coil, and is given by:

$${Q}_{s} = \frac{2 π {f}_{o} {L}_{s}}{{R}_{s}} \qquad ( 4 )$$

The inductance of the coil on the SCBC is determined based on the number of turns, inner and outer diameter, shape of the inductor, and spacing between lines as well as the permeability of the flexible substrate [10]. One ring of indium tin oxide (ITO) with an inner diameter of 10 mm, outer diameter of 10.076 mm, and linewidth 1 μm with spacing 10 μm results in 300 nH inductance. If the capacitance of the resonator is 460 pF, the resonance frequency will be 13.5 MHz. When the SCBC is placed on the cornea, the impedance of the eyeball acts as an electrical load for the resonator, shifting the resonance frequency, amplitude, phase angle, and/or quality factor. The reader unit will interrogate the changes in the resonator to obtain the reactance and conductance of the tissue. The separation between the SCBC and the reader as well as the operating frequency will determine the coupling factor, k.

The electrodes, coil for the inductor, and conductive plates for the capacitor may be formed using transparent and conductive thin film layers, such as ITO. The electrical components may be formed near the edge of the SCBC so as not to limit the field of view of the patient. Transparent electrodes will allow light to pass through the coil and will be almost unnoticeable; therefore, the contact lens will be comfortable for the wearer as well as cosmetically acceptable.

The soft contact lens may be formed by pouring PDMS mixture, including precursor and curing agents, into a mold and curing it to polymerize and form the soft contact lens. The mold will determine the curvature, diameter, and thickness of the contact lens. It will also form two holes in the soft contact lens for the electrical pads 459, 461 to pass through to make contact with the surface of the eyeball when the contact lens is placed in the eye. Shadow masks and magnetron sputtering may be used to form electrical components on the contact lens. In one embodiment, an initial deposit of a 100 nm-thick ITO through a first mask will be made to form a spiral conductor coil for the inductor 451 and the bottom electrodes 454a, 454b for the capacitor (not shown). A layer of high dielectric insulator (e.g., titanium oxide) may be deposited through a second shadow mask for the capacitor. The capacitor and/or any other surface mounted components may be covered with another layer to smooth out the overall contour of the lens and make it more comfortable to wear. Another layer of ITO may be deposited through a third shadow mask to complete the capacitor and electrical connections. A 100-nm thickness of polymer (e.g., parylene C) may be deposited on the surface of the contact lens to electrically insulate the components from fluids in the eye, and only the pads will be in contact with eye in the opposite side via through-holes in the PDMS.

The reader may be electromagnetically coupled with the SCBC to transfer energy and to read the impedance parameters of the eyeball. The oscillator of the reader unit may be configured to produce a pulsed sinusoidal output signal. Also, the reader unit may include an analog-to-digital converter, local memory, and a microcontroller to control the system and store data. The SCBC will not have any battery, power source or electronic chips. The power for the SCBC will be provided from the reader through the coupled inductors on the reader and the SCBC 450.

For characterization, the inductor, capacitor, and internal resistance of the SCBC may be measured using a precision LCR meter prior to use to obtain accurate values for those components. In addition, frequency response, electronic noises, leakage current in the capacitor, power dissipation, operating range, and coupling factor may be measured experimentally. For example, these parameters may first be measured without any electrical load (open circuit across the electrodes 459, 461) of the SCBC. Then, one or more electrical loads may be applied across the electrodes 459, 461 (e.g., with capacitors, resistors, and inductors) to calibrate the SCBC prior to use. Further, the impedance and electrical parameters of the test load may be measured with the reader module in the wireless mode to calibrate the system at various distance. The distance between the reader and the SCBC may be changed while taking measurements to determine the coupling factor and power transmission prior to use on a patient.

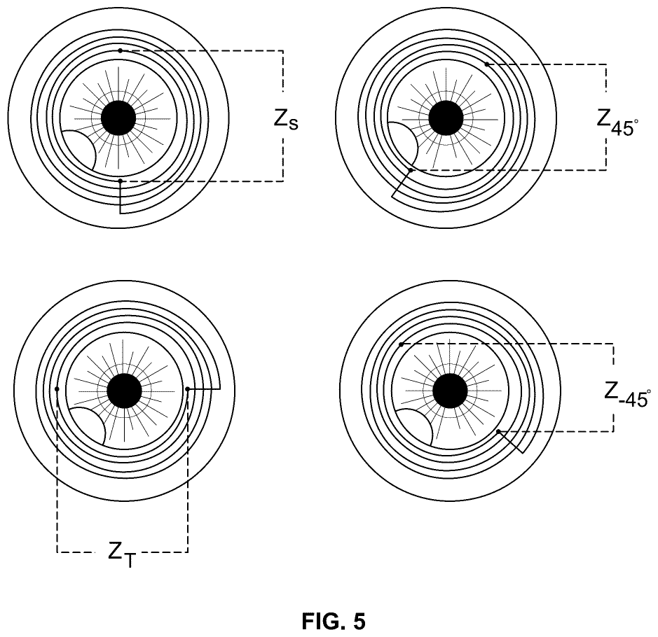

The orientation of the SCBC on the eye determines the location of impedance measurement. With reference to FIG. 5, rotating the SCBC on the eye to different orientations will allow measuring impedance on various planes (sagittal, 45°, transverse, and −45° plane). Data from the impedance measurement at multiple different angles may be analyzed to better determine the precise location of any electrical abnormality in the eye similar to mechanisms for Electrical Impedance Tomography [11]. Ciliary body tumors tend to significantly change the current distribution and impedance due to proximity to the SCBC.

A miniaturized camera may be installed on the reader module to determine the exact orientation of the contact lens on the eye, and impedance data will be assigned to the correct orientation.

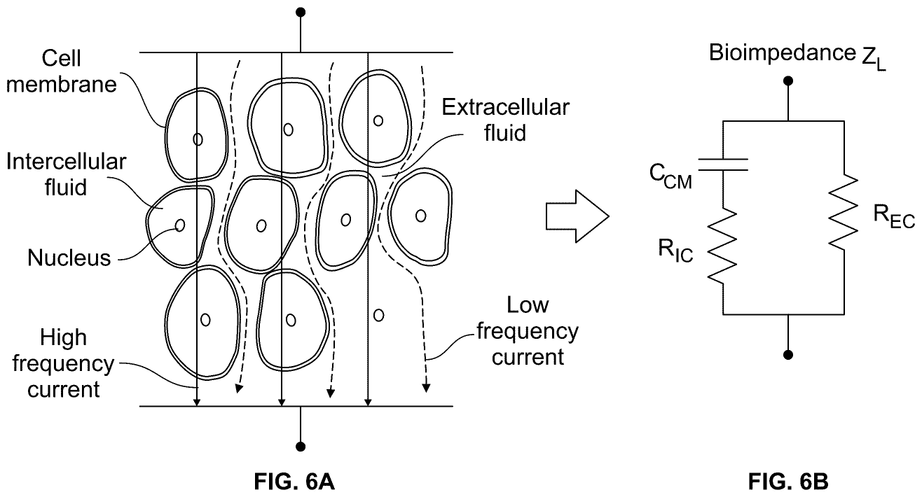

FIG. 6A shows the general structure of an eyeball at the cellular level (composed of cells and extracellular fluid (ECF), while FIG. 6B show the equivalent electrical circuit model of complex electrical impedance (Z_{L}). The intracellular fluid (ICF) is modeled by resistance R_{IC} and the ECF is modeled by resistance R_{EC} and contribute to the resistance of the overall circuit. The cell membranes form a bioelectric capacitance responsible for electrical reactance of the tissue [1], [12], [13]. The capacitive reactance of the cell membranes is modelled by capacitance X_{CM} and is inversely proportional to the frequency per

$${X}_{CM} = \frac{1}{2 π f {C}_{CM}} \qquad ( 5 )$$

Therefore, high-frequency currents will pass through the ECF, cell membranes, and ICF. The capacitances of the cell membranes around the cells exhibit large reactance at low frequencies, and, as a result, low-frequency currents mostly do not enter cells or pass through the ECF with resistance R_{EC}.

Finite element analysis (FEA) may be used to simulate the electrical properties of human eyes with tumors in the uveal tract to generate a robust database for readings indicative of uveal melanoma, and to validate the sensitivity needed to measure tumors present in different parts of the eye, e.g., the iris, ciliary body, and choroid. Reactance and conductance of the eyeballs as well as current density and voltage may be measured at various frequencies to find the optimal ranges for both for detecting abnormalities within the uveal tract.

The maximum power of the reader module may be limited to meet IEEE 1 g and 10 g specific absorption rate (SAR) of 1.6 W.kg-1 and 2 W.kg-1 for tissues [14]. The power requirement for the contact lens (<10 μW) is securely in the safe zone, since 100 mW was shown to be safe for eyes [15]. Maximum electrical current in the eyeball may be limited to 10 μA, which is safe for biological tissue (100 μA was safely used for electrical impedance tomography) [13].

In some embodiments, the SCBC may be a disposable device. However, the electrical components inside the SCBC will be packaged between two layers of protective polymers (e.g., PDMS, parylene-C) so the SCBC can be cleaned with a standard sterilization for soft contact lenses and can be reused, if so desired.

The SCBC offers many advantages and features. For instance, it provides non-invasive recording the electrical impedance of the eye. It also enables patient-driven monitoring of the electrical impedance of the eye over extended periods of time. The contact lens in conjunction with a handheld electronic reader (e.g., a smart phone) will allow long-term monitoring of the eye. This will provide an accessible tool to monitor the lesions outside of a clinical setting.

Furthermore, it can locate the site of the abnormalities in the eye and facilitate identification of the tumor type by placing the SCBC on the cornea at various angles to determine the impedance at different sites on the eyeball and locate the abnormalities.

Yet further, it is wireless and does not need to be removed from the eye to read the data.

Also, it is battery free. The SCBC will not have any batteries for storing electrical energy. The contact lens with a passive electrical resonator will receive electrical power from the reader. The resonator on the SCBC will be electromagnetically coupled with the reader.

Moreover, it is chip-less. The SCBC will not have any electronic chips and will only communicate with the reader through a passive inductor. This will significantly reduce the thickness, complexity, and cost of the SCBC.

Additionally, it is comfortable and cosmetically acceptable. Transparent electrodes will allow light to pass through the lens, and electrical components on SCBC will be unnoticeable. We will implement a thin layer of ITO on the contact lens to form transparent inductors and electrodes.

Even more, it will promote detection of electrical abnormalities associated with lesions in the ciliary body. Ciliary body melanoma is much more difficult to visualize. The lesions in the ciliary body are closer to the electrodes on the contact lens; therefore, they significantly affect the electric current density, and the device is much more sensitive to melanoma in the ciliary body.

The SCBC enables studying the correlation of electrical impedance with the presence of ocular tumors, including UM and PR. It further enables monitoring of individuals at a high risk of UM and PR. SCBC could be used outside of clinics and record data for analysis by the specialists. This will allow detection of any abnormalities in the very early stage and facilitate early treatment.

In certain embodiments, the device may be produced in the form of a wearable apparatus, such as a ring, smart watch or patch that takes continuous readings of the subject/patient. In some of these embodiments, the relevant data indicative of a condition or potential condition may be a change over a period of time in any of the aforementioned parameters related to tissue impedance. The period of time may be of any duration (e.g., from days to years).

In other embodiments, the device may not be a wearable device, but may be used by a patient in an at-home type scenario to screen for certain conditions. For example, a patient may observe a new mole on their skin and selectively use the device to take measurements on the new mole and on a pre-existing mole known to be healthy to measure the difference electrical impedance properties as a rough screening for cancer or other conditions. If the device measurements indicate that the new mole has different electrical characteristics than the pre-existing mole, the patient is advised to see a dermatologist.

In yet other embodiments, the device may be used clinically by a physician for the same purpose. If the data as to the expected changes in tissue impedance parameters is sufficiently robust and consistent, the device can be used to directly diagnose said condition. Merely as one example, the SCBC may be used to provide complementary data to ocular oncologists in combination with existing multimodal imaging strategies to assist in monitoring and diagnosing intraocular tumors.

Furthermore, this technology may be applied more broadly than the few examples provided hereinabove, such as for screening at-risk individuals with retinal detachment.

Having thus described a few particular embodiments of the invention, various alterations, modifications, and improvements will readily occur to those skilled in the art. Such alterations, modifications and improvements as are made obvious by this disclosure are intended to be part of this description though not expressly stated herein, and are intended to be within the spirit and scope of the invention. Accordingly, the foregoing description is by way of example only, and not limiting. The invention is limited only as defined in the following claims and equivalents thereto.

Although features and elements are described above in particular combinations, one of ordinary skill in the art will appreciate that each feature or element can be used alone or in any combination with the other features and elements. In addition, the methods described herein may be implemented in a computer program, software, or firmware incorporated in a computer readable medium for execution by a computer or processor. Examples of non-transitory computer-readable storage media include, but are not limited to, a read only memory (ROM), random access memory (RAM), a register, cache memory, semiconductor memory devices, magnetic media such as internal hard disks and removable disks, magneto-optical media, and optical media such as CD-ROM disks, and digital versatile disks (DVDs).

Moreover, in the embodiments described above, processing platforms, computing systems, controllers, and other devices containing processors are noted. These devices may contain at least one Central Processing Unit (“CPU”) and memory. In accordance with the practices of persons skilled in the art of computer programming, reference to acts and symbolic representations of operations or instructions may be performed by the various CPUs and memories. Such acts and operations or instructions may be referred to as being “executed,” “computer executed” or “CPU executed.”

One of ordinary skill in the art will appreciate that the acts and symbolically represented operations or instructions include the manipulation of electrical signals by the CPU. An electrical system represents data bits that can cause a resulting transformation or reduction of the electrical signals and the maintenance of data bits at memory locations in a memory system to thereby reconfigure or otherwise alter the CPU's operation, as well as other processing of signals. The memory locations where data bits are maintained are physical locations that have particular electrical, magnetic, optical, or organic properties corresponding to or representative of the data bits. It should be understood that the exemplary embodiments are not limited to the above-mentioned platforms or CPUs and that other platforms and CPUs may support the provided methods.

The data bits may also be maintained on a computer readable medium including magnetic disks, optical disks, and any other volatile (e.g., Random Access Memory (“RAM”)) or non-volatile (e.g., Read-Only Memory (“ROM”)) mass storage system readable by the CPU. The computer readable medium may include cooperating or interconnected computer readable medium, which exist exclusively on the processing system or are distributed among multiple interconnected processing systems that may be local or remote to the processing system. It is understood that the representative embodiments are not limited to the above-mentioned memories and that other platforms and memories may support the described methods.

In an illustrative embodiment, any of the operations, processes, etc. described herein may be implemented as computer-readable instructions stored on a computer-readable medium. The computer-readable instructions may be executed by a processor of a mobile unit, a network element, and/or any other computing device.

There is little distinction left between hardware and software implementations of aspects of systems. The use of hardware or software is generally (but not always, in that in certain contexts the choice between hardware and software may become significant) a design choice representing cost vs. efficiency tradeoffs. There may be various vehicles by which processes and/or systems and/or other technologies described herein may be effected (e.g., hardware, software, and/or firmware), and the preferred vehicle may vary with the context in which the processes and/or systems and/or other technologies are deployed. For example, if an implementer determines that speed and accuracy are paramount, the implementer may opt for a mainly hardware and/or firmware vehicle. If flexibility is paramount, the implementer may opt for a mainly software implementation. Alternatively, the implementer may opt for some combination of hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples may be implemented, individually and/or collectively, by a wide range of hardware, software, firmware, or virtually any combination thereof. Suitable processors include, by way of example, a general purpose processor, a special purpose processor, a conventional processor, a digital signal processor (DSP), a plurality of microprocessors, one or more microprocessors in association with a DSP core, a controller, a microcontroller, Application Specific Integrated Circuits (ASICs), Application Specific Standard Products (ASSPs); Field Programmable Gate Arrays (FPGAs) circuits, any other type of integrated circuit (IC), and/or a state machine.

The present disclosure is not to be limited in terms of the particular embodiments described in this application, which are intended as illustrations of various aspects. Many modifications and variations may be made without departing from its spirit and scope, as will be apparent to those skilled in the art. No element, act, or instruction used in the description of the present application should be construed as critical or essential to the invention unless explicitly provided as such. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods or systems.

It is also to be understood that the terminology used herein is for the purpose of describing particular embodiments only, and is not intended to be limiting.

In certain representative embodiments, several portions of the subject matter described herein may be implemented via Application Specific Integrated Circuits (ASICs), Field Programmable Gate Arrays (FPGAs), digital signal processors (DSPs), and/or other integrated formats. However, those skilled in the art will recognize that some aspects of the embodiments disclosed herein, in whole or in part, may be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein may be distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a CD, a DVD, a digital tape, a computer memory, etc., and a transmission type medium such as a digital and/or an analog communication medium (e.g., a fiber optic cable, a waveguide, a wired communications link, a wireless communication link, etc.).

The herein described subject matter sometimes illustrates different components contained within, or connected with, different other components. It is to be understood that such depicted architectures are merely examples, and that in fact many other architectures may be implemented which achieve the same functionality. In a conceptual sense, any arrangement of components to achieve the same functionality is effectively “associated” such that the desired functionality may be achieved. Hence, any two components herein combined to achieve a particular functionality may be seen as “associated with” each other such that the desired functionality is achieved, irrespective of architectures or intermediate components. Likewise, any two components so associated may also be viewed as being “operably connected”, or “operably coupled”, to each other to achieve the desired functionality, and any two components capable of being so associated may also be viewed as being “operably couplable” to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, where only one item is intended, the term “single” or similar language may be used. As an aid to understanding, the following appended claims and/or the descriptions herein may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”). The same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” Further, the terms “any of” followed by a listing of a plurality of items and/or a plurality of categories of items, as used herein, are intended to include “any of,” “any combination of,” “any multiple of,” and/or “any combination of multiples of” the items and/or the categories of items, individually or in conjunction with other items and/or other categories of items. Moreover, as used herein, the term “set” or “group” is intended to include any number of items, including zero. Additionally, as used herein, the term “number” is intended to include any number, including zero.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein may be readily broken down into a lower third, middle third and upper third, etc. As will also be understood by one skilled in the art all language such as “up to,” “at least,” “greater than,” “less than,” and the like includes the number recited and refers to ranges which can be subsequently broken down into subranges as discussed above. Finally, as will be understood by one skilled in the art, a range includes each individual member. Thus, for example, a group having 1-3 items refers to groups having 1, 2, or 3 items. Similarly, a group having 1-5 items refers to groups having 1, 2, 3, 4, or 5 items, and so forth.

Moreover, the claims should not be read as limited to the provided order or elements unless stated to that effect. In addition, use of the terms “means for” in any claim is intended to invoke 35 U.S.C. § 112, ¶6 or means-plus-function claim format, and any claim without the terms “means for” is not so intended.

Although the invention is illustrated and described herein with reference to specific embodiments, the invention is not intended to be limited to the details shown. Rather, various modifications may be made in the details within the scope and range of equivalents of the claims and without departing from the invention.

Throughout the disclosure, one of skill understands that certain representative embodiments may be used in the alternative or in combination with other representative embodiments.

### REFERENCES

[1]. A. A. Pathiraja et al., J. Transl. Med., vol. 18, no. 1, pp. 1-11, 2020.
[2]. V. Sturm and G. Richard, Klin Monbl Augenheilkd, vol. 224, no. 10, pp. 770-774, 2007.
[3]. C. S. M. Lau, I. Lee, and R. S. Chamberlain, ‘Trends in incidence, survival, and management of uveal
   melanoma: database (1973-2012),” pp. 2113-2119, 2016.
[4]. B. A. Krantz, et al., Clinical Ophthalmology, vol. 11. pp. 279-289, 2017.
[5]. S. Kaliki and C. L. Shields, Eye, vol. 31, no. 2, pp. 241-257, 2017.
[6]. E. Weis et al., Curr. Oneal., vol. 23, no. 1, pp. e57-e64, 2016.
[7]. A. M. Lane, I. K. Kim, and E. S. Gragoudas, JAMA Ophthalmol., vol. 136, no. 9, pp. 981-986, 2018.
[8]. M. M. Bras, et al., Front. Cell Dev. Biol., vol. 8, no. February, pp. 1-19, 2020.
[9]. C. L. Shields et al., Arch. Ophthalmol., vol. 127, no. 8, pp. 989-998, 2009.
[10]. S.S. Mohan, et. al., IEEE J. Solid-State Circuits, vol. 34, no. 10, pp. 1419-1420, 1999.
[11]. D. M. V. Cherepenin, etal., Physiol. Meas., vol. 22, pp. 8-18, 2001.
[12]. S. F. Khalil, M. S. Mohktar, and F. Ibrahim, Sensors, vol. 14, no. 6, pp. 10895-10928, 2014.
[13]. T. K. Bera, J. Med. Eng., vol. 2014, pp. 1-28, 2014.
[14]. D. B. Ahire and V. J. Gond, Proc.—Int. Conf. Trends Electron. Informatics, ICEI 2017, vol. 2018-Janua, pp. 135-140, 2018.
[15]. M. Yuan et al., Adv. Intel!. Syst., vol. 2, no. 4, p. 1900190, 2020.

1. A patch, for measuring electrical impedance in biological tissue, comprising:
a flexible substrate;
a resonator circuit on the flexible substrate comprising;
(a) an inductor; and
(b) a capacitor; and
first and second electrical contacts electrically connected to the resonator, which are exposed on a surface of the patch, for making electrical contact with the biological tissue.

2. The patch of claim 1 wherein the inductor and the capacitor of the resonator circuit are electrically coupled in parallel with each other and the first and second electrical contacts are electrically coupled in parallel with the resonator circuit.

3. The patch of claim 2 wherein the flexible substrate is formed of a polyimide.

4. The patch of claim 3 further comprising a first flexible insulating layer disposed on one side of the flexible substrate and a second flexible insulating layer disposed on an opposing side of the flexible substrate, and wherein the first and second electrical contacts extend through the second insulating layer to provide electrical contact with biological tissue when the patch is positioned with the second flexible insulator in contact with biological tissue.

5. The patch of claim 1 wherein the inductor is printed on the flexible substrate and the capacitor is surface mounted to the flexible substrate.

6. The patch of claim 1 wherein the patch is formed on a contact lens for an eye.

7. The patch of claim 1 wherein the flexible substrate is formed of polydimethylsiloxane (PDMS).

8. The patch of claim 7 wherein the inductor, capacitor, and first and second electrical contacts are located toward the edge of the contact lens such that they will not obstruct the vision of a subject earing the contact lens.

9. A system for measuring impedance in biological tissue, comprising:
a patch comprising:
a flexible substrate;
a first resonator circuit on the flexible substrate comprising a first inductor and a first capacitor electrically coupled in parallel; and
first and second electrical contacts electrically connected in parallel with the resonator circuit, which are exposed on a surface of the patch, for making electrical contact with the biological tissue; and
a reader, comprising:
a second resonator circuit comprising a second inductor and a second capacitor electrically coupled in parallel;
an electric oscillator coupled to provide for periodic electrical signal across the second resonator circuit,
a voltage monitoring circuit coupled to read a voltage across the second resonator; and
wherein the reader is configured such that the first inductor will inductively couple with the second inductor to transfer energy from the oscillator to the first resonator circuit via the inductive coupling when the reader is positioned in proximity to the patch.

10. A method of detecting a biological condition of biological tissue comprising:
providing a patch comprising a flexible substrate, a first resonator circuit on the flexible substrate comprising a first inductor and a first capacitor electrically coupled in parallel, and first and second electrical contacts electrically connected in parallel with the resonator circuit, which are exposed on a surface of the patch, for making electrical contact with the biological tissue;
providing a reader comprising a second resonator circuit comprising a second inductor and a second capacitor electrically coupled in parallel, an electric oscillator coupled to provide for periodic electrical signal across the second resonator circuit, and a voltage monitoring circuit coupled to read a voltage across the second resonator;
disposing the patch such that the first and second electrical contacts are in electrical contact with a first biological tissue;
positioning the reader in proximity of the patch such that the first inductor and the second inductor inductively couple;
applying a periodic signal across the second resonator circuit;
measuring a first voltage across the second resonator; and
evaluating the first measured voltage to determine a biological condition of the first biological tissue.

11. The method of claim 10 wherein the periodic signal is sinusoidal.

12. The method of claim 11 wherein the sinusoidal signal is pulsed on and off periodically.

13. The method of claim 10 wherein a frequency of the sinusoidal signal is varied over time over a range of frequencies and wherein the evaluating of the measure voltage includes determining a resonance frequency of the second resonator.

14. The method of claim 10 wherein the determining the resonance frequency of the first resonator comprises determining the frequency in the range that caused the maximum measured voltage across the second resonator.

15. The method of claim 10 wherein the evaluating of the measure voltage includes determining one of an amplitude of the measured voltage and a phase shift of the measured voltage.

16. The method of claim 13 further comprising:
disposing the patch such that the first and second electrical contacts are in electrical contact with a second biological tissue;
positioning the reader in proximity of the patch such that the first inductor and the second inductor inductively couple;
applying the periodic signal across the second resonator circuit; and
measuring a second voltage across the second resonator; and
wherein evaluating the measured voltage to determine a biological condition of the first biological tissue comprises comparing the first and second measured voltages.

17. The method of claim 16 wherein the evaluating the measured voltage to determine a biological condition comprises comparing the phases of the first and second measured voltages.

18. The method of claim 16 wherein the evaluating the measured voltage to determine a biological condition comprises determining a difference in amplitude between the first and second measured voltages.

19. The method of claim 16 wherein the evaluating the measured voltage to determine a biological condition comprises determining a difference in a resonance frequency of the first and second measured voltages.

20. The method of claim 16 wherein the evaluating the measured voltage to determine a biological condition comprises determining a difference in a frequency of the first and second measured voltages.
